SEQUENCE LISTING (5 sequences)

SEQ ID NO: 1 - DNA, 6458 bp, Artificial sequence
  Plasmid pcDNA3.1hCD5.6Ala7
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagccaccatgcccatggggtctctgcaaccgctggccaccttgtacctgctggggatgctggtcgcttcctgcctcggaaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttggacagttttgtacatattgtcgacatgttcatcaacacttcggatcccaaatctgctgacaaaactcacacatgcccaccgtgcccagcacctgaactcctggggggaccgtcagtcttcctcttccccccaaaacccaaggacaccctcatgatctcccggacccctgaggtcacgtgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 2 - DNA, 7464 bp, Artificial sequence
  Plasmid pMG10Ala7
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgtaagctgcaataaacaatcattattttcattggatctgtgtgttggttttttgtgtgggcttgggggagggggaggccagaatgactccaagagctacaggaaggcaggtcagagaccccactggacaaacagtggctggactctgcaccataacacacaatcaacaggggagtgagctggatcgagctagagtccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggacatgggctcttctccggtagcggcggagcttctacatccgagccctgctcccatgcctccagcgactcatggtcgctcggcagctccttgctcctaacagtggaggccagacttaggcacagcacgatgcccaccaccaccagtgtgccgcacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcacccgggggatccttcgaacgtagctctagccaccatgcccatggggtctctgcaaccgctggccaccttgtacctgctggggatgctggtcgcttcctgcctcggaaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttggacagttttgtacatattgtcgacatgttcatcaacacttcggatcccaaatctgctgacaaaactcacacatgcccaccgtgcccagcacctgaactcctggggggaccgtcagtcttcctcttccccccaaaacccaaggacaccctcatgatctcccggacccctgaggtcacgtgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 3 - DNA, 1113 bp, Artificial sequence
  DNA for mutated IL-15/Fc with CD5 leader
atgcccatggggtctctgcaaccgctggccaccttgtacctgctggggatgctggtcgcttcctgcctcggaaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttggacagttttgtacatattgtcgacatgttcatcaacacttcggatcccaaatctgctgacaaaactcacacatgcccaccgtgcccagcacctgaactcctggggggaccgtcagtcttcctcttccccccaaaacccaaggacaccctcatgatctcccggacccctgaggtcacgtgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 4 - protein, 370 aa, Artificial sequence
  Amino acid sequence of human CRB-15 with CD5 leader
MPMGSLQPLATLYLLGMLVASCLGNWVNVISDLKKIEDLIQSMHIDATLYTESDVHPSCKVTAMKCFLLELQVISLESGDASIHDTVENLIILANNSLSSNGNVTESGCKECEELEEKNIKEFLDSFVHIVDMFINTSDPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 371 aa, Artificial sequence
  Amino acid sequence of murine IL-15/Fc (human mutated IL-15, murine IgG2A) with CD5 leader
MPMGSLQPLATLYLLGMLVASCLGNWVNVISDLKKIEDLIQSMHIDATLYTESDVHPSCKVTAMKCFLLELQVISLESGDASIHDTVENLIILANNSLSSNGNVTESGCKECEELEEKNIKEFLDSFVHIVDMFINTSDPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

(19) United States
(12) Patent Application Publication
(10) Pub. No.: US 2009/0105455 A1
(43) Pub. Date: Apr. 23, 2009

(54) Purified interleukin-15/fc fusion protein and preparation thereof

(76) Inventor: Andreas Herrmann, Koeln (DE)

Correspondence Address: CLARK & ELBING LLP, 101 FEDERAL STREET, BOSTON, MA, 02110, US

(21) Appl. No.: 10/592,332

(22) PCT Filed: Apr. 13, 2005
(86) PCT No.: PCT/EP2005/003887

§ 371 Date: Jul. 11, 2007

(30) Foreign Application Priority Data
Apr. 14, 2004 (EP) 04008906.2

(51) Int. Cl. C07K 14/54; C07K 1/00

(52) U.S. Cl. 530/351; 530/413

(57) ABSTRACT

The present invention relates to a process for purifying an interleukin-15/Fc fusion protein from a composition, which process comprises a) applying the composition to an affinity chromatography column and eluting a first IL-15/Fc eluate from the column and b) applying the eluate of step a) to an ion exchange chromatography column and eluting a second IL-15/Fc eluate from the column; and to a purified interleukin-15/Fc fusion protein and a composition, in particular a pharmaceutical composition, comprising such a fusion protein.

The present invention relates to a process for purifying an interleukin-15/Fc fusion protein from a composition, which process comprises a) applying the composition to an affinity chromatography column and eluting a first IL-15/Fc eluate from the column and b) applying the eluate of step a) to an ion exchange chromatography column and eluting a second IL-15/Fc eluate from the column; and to a purified interleukin-15/Fc fusion protein and a composition, in particular a pharmaceutical composition, comprising such a fusion protein.

The transplantation of organs or tissues has become the standard method and, in numerous cases, the only lifesaving treatment of many life-threatening diseases. However, difficulties frequently arise with regard to rejections by the recipient organism, which are caused by an immune response to the foreign cell surface antigens of the transplant. One therapeutic approach of suppressing rejection is to suppress the humoral or cellular immune response by means of immunosuppressives, in particular by antagonistic interleukin-15 (IL-15) or interleukin-2 (IL-2) antibodies or agonists. Various therapies using antibodies to IL-15 or IL-2 molecules have been described previously. An example of an effective IL-15 antagonist is a fusion protein consisting of an N-terminal mutated or unmutated IL-15 fragment and a C-terminal Fc fragment (WO 97/41232; Kim et al. (1998) J. Immunol. 160: 5742-5748).

In order to be able to employ the fusion proteins successfully as pharmaceuticals, it is necessary to be able to produce them with very high purity on a large scale and to store them in a stable manner. Generally, proteins which do not occur naturally are produced by means of genetic engineering processes. To this end, the said proteins are produced in cell cultures, with either mammalian or bacterial cell lines having been genetically modified so as for a recombinant plasmid containing the gene of the peptide of interest to be introduced into the cell lines, in order to produce the protein. These cell lines are then cultured under suitable conditions in the presence of complex culture media which contain, for example, sugars, amino acids, growth factors, salts, sera from different animals etc. The protein of interest subsequently needs to be separated from the components of the medium, the metabolic products of the cells and other contaminations, until a purity is achieved which is sufficient for use as a therapeutic agent.

Processes for purifying proteins from cell cultures are well known to the skilled worker. Some proteins are released by the cell lines directly into the surrounding medium, others are retained inside the cell. The latter require to first disrupt the cell, which is made possible by a multiplicity of processes such as, for example, mechanical shearing, osmotic shock or enzymic treatment. In this case, the homogenate includes the entire cell contents and additional processes are required in order to remove subcellular fragments. The latter processes include, for example, differential centrifugation or filtration. If the proteins are obtained directly from the supernatant, such steps may also be necessary in order to remove parts of dead cells or the like. Thereafter, proteins are usually purified further by a combination of various chromatographic techniques. These techniques separate mixtures of proteins depending on their size, charge, hydrophobicity or affinity for particular substrates. For each of these techniques, a number of column materials are available which are used depending on the protein of interest. The aim of any chromatography is for the protein of interest to exhibit a migration behaviour on the column, which is different from that of the contaminations, and thus to elute at a different time than the latter.

It is an object of the present invention to provide an IL-15/Fc fusion protein as pure as possible in a storable form.

The object was achieved by a process for purifying IL-15/Fc from a composition, which process comprises the following steps:
a) applying the composition to an affinity chromatography column and eluting a first IL-15/Fc eluate from the column and
b) applying the eluate of step a) to an anion exchange chromatography column and eluting a second IL-15/Fc eluate from the column.

The IL-15/Fc fusion protein (IL-15/Fc), as used herein, is a fusion protein comprising two fusion moieties, namely an IL-15 component and an Fc component. Recombinant proteins which comprise a fusion moiety of an immunoglobulin in addition to a functional protein are described, for example, in Capon et al. (U.S. Pat. No. 5,428,130).

Preference is given to a fusion protein which consists of an N-terminal mutated or unmutated IL-15 part and a C-terminal Fc part. Such proteins are disclosed, for example, in WO 97/41232 and Kim et al. (1998, J. Immunol. 160: 5742-5748).

The IL-15 part of the fusion protein mediates selective binding to the IL-15 receptor (IL-15R) which is expressed on activated T cells, for example. The IL-15 part may therefore be both a naturally occurring IL-15 and a mutant thereof.

In a more preferred embodiment, the IL-15 component is wild-type IL-15. In this connection, the IL-15 may be an IL-15 of any species such as, for example, mice, rats, guinea pigs, rabbits, cattle, goats, sheep, horses, pigs, dogs, cats or monkeys, preferably humans. Included are also different splice variants and naturally occurring variants. Particular preference is given here to nucleic acids of mammals, in particular the human or murine form of the nucleic acids.

IL-15 mutants include IL-15 components which, compared with the naturally occurring IL-15, have a mutation such as, for example, one or more deletions, insertions or substitutions or combinations thereof. The IL-15 variant used, however, must enable the IL-15/Fc fusion protein to bind to the IL-15R. This could be checked, for example, in a radio ligand binding assay using labelled IL-15 and membranes or cells having IL-15 receptors (Carson W E et al., 1994, J Exp Med., 180(4): 1395-1403).

In a preferred embodiment, the mutant may have an action like IL-15 (IL-15 component with agonist action) and whose activity, in comparison with IL-15, may be at the same, a reduced or even an increased level. A test system which may be used for IL-15/Fc fusion proteins having an IL-15 component with agonist action is the stimulation of murine CTLL-2 cell proliferation by the said IL-15 component.

An IL-15 component has agonist action in accordance with the present invention, if the component has at least 10%, preferably at least 25%, more preferably at least 50%, still more preferably 100%, even more preferably 150% and most preferably at least 200% activity. Activity of an IL-15 component with agonist action means the percentage of stimulation of the response by the IL-15 component in comparison with stimulation by wild-type IL-15 (wild-type IL-15 corresponds to 100% activity). It is possible to use in the assays either the IL-15 component alone or the fusion protein.

For IL-15 components with agonist action, preference is given to conservative amino acid replacements, with a residue being replaced with another one having similar properties. Typical substitutions are substitutions within the group of aliphatic amino acids, within the group of amino acids with aliphatic hydroxyl side chain, within the group of amino acids with acidic radicals, within the group of amino acids with amide derivatives, within the group of amino acids with basic radicals or among the amino acids with aromatic radicals. Typical conservative and semi-conservative substitutions are the following:

| | Conservative | Semi-conservative |
|---|---|---|
| Amino acid | substitution | substitution |
| A | G; S; T | N; V; C |
| C | A; V; L | M; I; F; G |
| D | E; N; Q | A; S; T; K; R; H |
| E | D; Q; N | A; S; T; K; R; H |
| F | W; Y; L; M; H | I; V; A |
| G | A | S; N; T; D; E; N; Q |
| H | Y; F; K; R | L; M; A |
| I | V; L; M; A | F; Y; W; G |
| K | R; H | D; E; N; Q; S; T; A |
| L | M; I; V; A | F; Y; W; H; C |
| M | L; I; V; A | F; Y; W; C |
| N | Q | D; E; S; T; A; G; K; R |
| P | V; I | L; A; M; W; Y; S; T; C; F |
| Q | N | D; E; A; S; T; L; M; K; R |
| R | K; H | N; Q; S; T; D; E; A |
| S | A; T; G; N | D; E; R; K |
| T | A; S; G; N; V | D; E; R; K; I |
| V | A; L; I | M; T; C; N |
| W | F; Y; H | L; M; I; V; C |
| Y | F; W; H | L; M; I; V; C |

In another embodiment of the present invention, use is made of IL-15 components with antagonist action. Components of this type inhibit the action of IL-15 or binding of IL-15 to IL-15R, it being possible for the inhibition to be complete or only partial. A test system which may be used for IL-15/Fc fusion proteins which have an IL-15 component with antagonist action is the test system described in WO 97/41232 (BAF-BO3 cell proliferation assay). An IL-15 component has antagonist action in accordance with the present invention, if the component inhibits at least 10%, preferably at least 25%, more preferably at least 50% and most preferably at least 95% of the IL-15-mediated action or IL-15 binding to IL-15R. It is possible to employ in the assays either the IL-15 component alone or the fusion protein.

For IL-15 components with antagonist action, preference is given to non-conservative amino acid replacements, with a residue being replaced with another one having different properties. Preference is further given to these replacements taking place in regions of the molecule which are responsible for the interaction with IL-15-R or for signal transduction.

In a preferred embodiment, the IL-15 components with antagonist action used are the IL-15 mutants described in WO 97/41232 or an IL-15 component having a mutation at amino acid position 56 (aspartate; AAA21551). Most preference is given to mutants into which point mutations have been introduced at amino acid positions 149 and/or 156 of interleukin-15, replacing glutamine with aspartate in particular (see WO 97/41232).

In one embodiment it is also possible to combine the mutations described.

In one embodiment, the mutated IL-15 part of the fusion protein is at least 65%, preferably at least 70%, more preferably at least 85%, still more preferably at least 95% and most preferably at least 99%, identical to the wild-type IL-15, preferably to a human wild-type IL-15 (e.g. database of the National Center for Biotechnology Information, accession number AAA21551), or else other naturally occurring variants (e.g. the variants with accession numbers CAA63914 and CAA71044 of the database of the National Center for Biotechnology Information).

The second functional unit of the IL-15/Fc fusion protein is an Fc component. The Fc part means the constant (c=constant) fragment of immunoglobulins, which can be prepared by papain cleavage and whose amino acid sequence is highly conserved. The Fc fragment is the antibody fragment which usually does not bind any antigens. An Fc part according to the present invention means preferably also an immunoglobulin fragment as defined above which, in addition, also comprises the constant domains CH2 and CH3 besides the hinge region.

The Fc component is derived from the Fc part of any antibody, for example of an IgA, IgD, IgG, IgE or IgM, preferably of an IgM or an IgG, more preferably from an Fc part of the subclasses IgG1, IgG2, IgG3 and IgG4.

In a particular embodiment of the invention, the Fc part of the fusion protein is an Fc fragment of an immunoglobulin G (IgG), which lacks the light chains and heavy chains of the IgG-variable region.

Examples of IgGs which may be used are IgG1, IgG2, IgG2a, IgG2b, IgG3 and IgG4. Preference is given to human or murine IgG1.

It is possible to use for the present invention the entire Fc part of the antibody or only a part thereof. However, the said part of the Fc part should be designed preferably in such a way that the IL-15/Fc fusion protein has a longer half life of circulation in the blood than the IL-15 component without immunoglobulin component. This may be tested by administering to, for example injecting into the blood stream of, one or more experiment animals the fusion protein and the IL-15 component and comparing the half lives of circulation in the blood. A longer half life is indicated by an increase in half life, preferably by at least 10%, more preferably at least 20%, still more preferably at least 50% and most preferably at least 100%.

The Fc part may also be an Fc part having at least one mutation. The mutated Fc may be mutated in the manner described above for the IL-15 part.

In one embodiment, the mutated Fc part of the fusion protein is at least 65%, preferably at least 70%, more preferably at least 85%, still more preferably at least 95% and most preferably at least 99% identical to the Fc part of a murine or human wild-type immunoglobulin, preferably to the human IgG1-Fc or else naturally occurring variants.

In a preferred embodiment of the invention, the Fc moiety of the fusion protein is the native form or has conservative amino acid replacements and contains intact FcR- and/or complement-binding sites. The Fc moiety of the fusion protein may mediate both activation of the complement system and binding to Fc receptor-expressing cells and thus result in the depletion of the cells recognized by the IL-15 moiety of the fusion protein. The introduction of mutations, in particular of non-conservative amino acid replacements, at the amino acid positions which mediate complement activation and Fc-receptor binding makes it possible to switch off these functions. Examples of these mutations are those of the binding site for the Fc receptor (FcR) or the complement-binding sites (at amino acid positions 214, 356, 358 and/or 435 in the native human IgG1 or Leu 235, Glu 318, Lys 320 and/or Lys 322 in the native murine IgG2A). The replacement of amino acids at these positions usually results in a loss of the lytic and complement-activating function of the Fc moiety (WO 97/41232).

Still further preference is given to an embodiment in which the amino acid cysteine at position 4 of the hinge region of the human Fc moiety, more preferably of the human IgG1 (position 167 of human IgG1), has been replaced with alanine, for example in order to prevent intermolecular bridging and thus aggregation of the expressed IL-15/Fc fusion protein.

In another preferred embodiment the Fc part is the Fc part of human immunoglobulin IgG1 or of the murine immunoglobulin IgG2A, which, in addition to the hinge region, comprises the heavy-chain regions CH2 and CH3.

In the IL-15/Fc fusion protein, the IL-15 component is fused to the immunoglobulin component either directly or via a linker. The linker consists preferably of no more than 25 amino acids, more preferably of no more than 15 amino acids, still more preferably of no more than 10 amino acids and most preferably of 1, 2, 3, 4 or 5 amino acids.

In a preferred embodiment of the invention, the fusion protein is encoded by the nucleic acids of positions 905 to 2014 of SEQ ID NO. 1, of positions 1911 to 3020 of SEQ ID NO. 2 or SEQ ID NO. 3, the signal sequence (CD5) being encoded in each case by the first 74 nucleotides. However, it may also be a protein of SEQ ID NO. 4 or SEQ ID NO. 5. Protein whose sequence is at least approx. 60%, preferably approx. 75%, particularly preferably approx. 90% and in particular approx. 95% identical to any of the abovementioned sequences is also comprised, with the corresponding IL-15/Fc fusion protein binding to IL-15R and having an increased half life in the blood, compared with the corresponding IL-15/Fc fusion protein without immunoglobulin component (for test systems, see above).

The fusion protein may be produced by introducing a nucleic acid coding for the fusion protein into a cell and subsequently expressing the nucleic acid in the cell under suitable conditions. The fusion protein produced in this way may then be recovered either from the supernatant or from the cells themselves by means of the process according to the invention. This involves applying the composition containing the IL-15/Fc fusion protein to an affinity chromatography column and eluting a first IL-15/Fc eluate.

Affinity chromatography means a special form of adsorption chromatography in which groups with high affinity for and therefore high strength of binding to the substances to be removed are present on a support so that the said substances are able to be adsorbed preferentially and thus be removed from other substances.

The support may be, for example, a solid phase of a purification column, a discontinuous phase or discrete particles. A possible example of the solid phase is a column of porous glass or a silica column. In one embodiment of the invention, the solid phase may be coated with a reagent (such as, for example, glycerol) in order to suppress non-specific attachment of contaminants to the solid phase. In a preferred embodiment, the support material used for chromatography is Sepharose, in particular if protein A and/or protein G are used as ligands.

The binding partner to be isolated may be recovered by a) binding of ligand and binding partner to be recovered under suitable conditions, b) washing out non-binding substances, where appropriate, c) separating the binding partner from the ligand by generating conditions which no longer allow binding of the two molecules, for example changing the pH or ionic strength of the buffer solution.

In a preferred embodiment, the affinity chromatography is a protein-A chromatography using protein A, particularly preferably recombinant protein A, as ligand. Protein A is a bacterial cell-wall protein of Staphylococcus aureus, which has a specific affinity for the Fc region of immunoglobulins of the G class (IgG). Protein A has a molecular weight of 42 kDa (recombinant protein A: approx. 32 to 45 kDa) and high pH stability of from pH 2 to 10. Protein A is immobilized to the solid phase (support material). The binding affinity for the Fc part is a function of the pH and, after binding in the presence of neutral or slightly alkaline buffers, the immunoglobulins can be eluted with a decreasing pH gradient.

One example of a protein-A chromatography material is Prosep-A (BioProducing Ltd., UK) which consists of protein A bound to glass in a pore structure. Other suitable protein-A formulations are protein A Sepharose, e.g. Protein A Sepharose Fast Flow (Pharmacia) and Toyopearl 650 Protein A (TosoHaas).

Alternatively, the affinity chromatography may also be a protein-G chromatography in which the ligand used is protein G. Protein G is a surface protein from Streptococci of the G group and has an affinity spectrum different from that of protein A. The affinity of the IgG antibodies for protein A differs from that for protein G so that it is possible for the skilled worker to select a suitable affinity chromatography, depending on the Fc part used.

It is furthermore also possible to use antisera of heterologous species, which are directed to the Fc part or else the IL-15 part of the fusion protein.

To carry out step a) of the process according to the invention, the column for purification may moreover be equilibrated, for example, with a suitable buffer solution. A buffer is a buffered solution which stabilizes the pH owing to its acid-based conjugates. The buffer used for equilibrating the column is preferably an isotonic buffer whose pH is usually in the range from approximately 6-8. A possible example of an equilibration buffer is 20 mmol/l Tris, 25 mmol/l NaCl, 25 mmol/l EDTA, pH 7.5.

The composition containing the IL-15/Fc fusion protein in a suitable buffer is then applied in loading buffer to the column. In one embodiment, the compositions of equilibrium buffer and loading buffer are identical. The column is subsequently washed, where appropriate, with a suitable washing buffer and, thereafter, the IL-15/Fc fusion protein is eluted with an elution buffer of choice.

The optional washing step which may be carried out between loading of the column (applying the composition) and eluting serves to remove contaminants which are bound non-specifically to the solid phase. The aim is to elute as little fusion protein as possible from the solid phase. The washing step may usually be carried out with an isotonic solution such as, for example, 20 mmol/l Tris, 150 mmol/l NaCl pH 7.4, or else by lowering the pH to a lower value at which the fusion protein still binds, however, for example at pH 5.5.

The elution buffer is used in order to elute the IL-15-Fc fusion protein from the column. The pH of the elution buffer is preferably low, thus destroying the interaction between the ligand, in particular protein A, and the fusion protein. The pH of the elution buffer is preferably in the range from 2-5, more preferably in the range from 3-4. Examples of buffers controlling the pH in this range are phosphate, acetate, citrate and ammonium buffers and mixtures thereof. Preferred buffers are citrate and acetate buffers, with further preference being given to sodium citrate or sodium acetate buffers, and most preference being given to 0.1 mol/l citrate, pH 3.4. However, it is also possible to use elution buffers which have a high pH (e.g. pH 9 and higher) or other buffers which disturb the interaction between the ligand, in particular protein A, and the fusion protein.

The eluate of step a) is, where appropriate, diluted, applied, in step b), to an ion exchange chromatography column, a cation exchange chromatography column or, preferably, an anion exchange chromatography column, and a second IL-15/Fc eluate is eluted from the column.

In a preferred embodiment of the invention, the pH of the eluate of step a) is increased by adding a base or a buffer. This is preferably carried out immediately after elution, in order to prevent possible inactivation of the fusion protein. This may be carried out, for example, by adding one tenth of the volume of 1 mol/l Tris pH 8.0.

In ion exchange chromatography, proteins are separated based on their charge. Proteins consist of various amino acids whose side chains may usually carry, in addition to uncharged radicals, also acidic and basic radicals and which thus contribute to the total charge of the protein. At low pH, below the isoelectric point of the protein, the total charge is positive, due to protonation of the charged side chains, at higher pH it is negative, due to deprotonation. Since proteins carry a multiplicity of charged groups whose actual charge depends on the pH and also on the environment of the individual amino acid, separation according to charge is a powerful method of separating proteins. In a ion exchange chromatography, the pH is chosen so as to enable the protein of choice to bind to the matrix. In the case of anion exchangers, this pH is usually at least one pH unit above the isoelectric point of the protein (pH at which the protein has a net charge of 0), in the case of cation exchangers it is below the isoelectric point. Binding to the matrix takes place via electrostatic interactions. Proteins which do not bind to the matrix are washed out with buffer. The bound protein is eluted by adding salts, with preference being given to using NaCl. The presence of, for example, charged sodium and chloride ions stops the electrostatic interactions of proteins with the matrix so that the proteins are detached from the matrix.

Anion exchangers or basic ion exchangers are ion exchangers in which the cationic group is covalently bound to a solid insoluble matrix, while the neutralizing anion is bound only ionically and can therefore be replaced with other anions. The chromatography by means of anion exchanger is usually used for purifying negatively charged molecules. Examples of anion exchangers are aminoethyl, diethylaminoethyl, quaternary aminoethyl and quaternary ammonium groups, coupled to a polyacrylamide gel resin or to a hydrocarbon polymer resin such as, for example, cellulose or dextran.

The cation exchange chromatography too, works according to an analogous principle. Here, positively charged molecules to be purified are bound to a support matrix which contains negatively charged groups (e.g. carboxymethyl or sulphonic acid groups). Counterions used are normally sodium or potassium ions which are replaced with the positively charged sample molecules. Examples of cation exchangers are carboxymethyl, sulphoethyl, sulphopropyl, phosphate or sulphonate groups, coupled to a polyacrylamide gel resin or to a hydrocarbon polymer resin such as, for example, cellulose or dextran.

The column configuration may be, for example, the customary one with vertical flow or else one with radial flow.

Prior to applying the eluate of step a), the resin is usually equilibrated, suitably with a buffer having a pH in the range from 5-10, preferably 6-9, more preferably 7-8. A multiplicity of buffers is capable of maintaining the pH in this range. Each of these buffers is suitable for use, but currently preference is given to a Tris buffer, more preferably a buffer with 20 mmol/l Tris, pH 8.0.

After equilibration, the eluate of step a) is applied to the ion exchange resin, preferably the anion exchange resin. The column is then, where appropriate, washed and subsequently the IL-15-Fc fusion protein is eluted by increasing the ionic strength and/or changing the pH. The optional washing step normally makes use of the same buffer as that of the equilibration phase. However, it is also possible to use a washing buffer having a different, for example a higher, molarity, such as, for example, 20 mmol/l Tris, 200 mmol/l NaCl pH 8.0. The buffer concentration for chromatographic separation is usually, for example, at least 10 mmol/l in order to ensure sufficient buffer capacity. The ionic strength of the buffer is usually kept low (<5 mS/cm) so as not to affect binding of the proteins to the matrix. However, the ionic strength should not be so low that the protein denatures or precipitates. The buffer ion should have the same charge as the matrix, since an opposite charge affects the separation process and results in local pH disturbances. The elution may be carried out using a linear gradient, a step-by-step increase in the concentration of the electrolyte or carried out isocratically. Usually, final molarities of up to 0.5 mol/l, preferably up to 0.4 mol/l, more preferably up to 0.35 mol/l, are obtained, the electrolyte used, for example, being sodium chloride or else another electrolyte which has the same action.

The eluate of step a), having a preferred buffer concentration of 10-200 mmol/l, is applied to the equilibrated column. The pH of the eluate is adjusted to 5-9, preferably to 7.5-8.5. The pH may be adapted by means of suitable agents, for example by adding NaOH or another suitable base. Next, the column may be washed with buffer, preferably the equilibration buffer or washing buffer. The buffer concentration is between preferably 10 and 300 mmol/l. The pH is preferably in the range between 7.0 and 9.0, more preferably between 7.75 and 8.25.

In a preferred embodiment of anion exchange chromatography, the column employed is preferably a DEA-Sepharose column or a Q-Sepharose column, more preferably a Q-Sepharose column.

In a preferred embodiment, the anion exchange chromatography is carried out by means of FPLC (Fast Protein Liquid Chromatography) equipment or Äkta purifiers (e.g. Äkta Pilot or BioPilot, Amersham, UK). In the case of FPLC equipment, the ion exchange column is first equilibrated in the starting buffer and the protein mixture to be separated, which is in a sample loop, then is applied to the column. Proteins which do not bind to the column are eluted immediately. The ionic strength in the buffer is slowly increased by admixing buffer so that the proteins elute one-by-one from the column and are collected in a fraction collector.

In a preferred embodiment of the invention, the purification process according to the invention additionally comprises step c):
c) applying the eluate of step b) to a gel filtration column or to a hydrophobic interaction chromatography column and eluting a third IL-15/Fc eluate from the column.

The eluate of step b) is here purified by means of gel filtration or hydrophobic interaction chromatography.

Gel filtration is particularly suitable for separating proteins by means of chromatography whose stationary phase consists of swelled gel beads and which separates according to particle size: large particles migrate with the liquid past the gel beads, smaller ones are retained in the pores and are the last to appear in the eluate. The elution is usually carried out isocratically, i.e. with only one buffer without gradient. The composition of the buffer usually has no influence on the separation and depends on the requirements of the protein. Spherical polymers whose porosity depends on the degree of their crosslinking may be used as gel matrix. Examples thereof are Sephadex, Sepharose, Biogel A, Sephacryl and Biogel P. Smaller proteins of a suitable size can diffuse into the pores of these matrices. This slows down their flow rate through the gel. If the proteins are too large, they elute past the pores and with a higher flow rate through the gel. High-molecular-weight proteins thus elute before low molecular-weight proteins. The substance mixtures are separated according to different Stokes' radii which, in the case of globular proteins, are usually proportional to the molecular weight of the molecule. In order to obtain sufficient separation, the volume applied is preferably no more than 5%, more preferably no more than 1%, of the column volume. The concentration may preferably be up to 50 mg/ml, more preferably 25 mg/ml and even more preferably 10 mg/ml.

The stationary phase of the gel filtration chromatography column comprises a column material which preferably fractionates in the range between 5000-600 000 kDa. Preference is given to using Superdex 200, Superdex 75, Superose 6, Superose 12, Sephadex G 75, Sephacryl S-200 HR, Sephacryl S-300 HR or Sephacryl S-400 HR, more preferably Superdex 200, as column material.

The column is usually equilibrated, prior to applying the eluate of step b), with equilibration buffer, for example 50 mmol/l sodium phosphate, pH 7.0, 150 mmol/l sodium chloride. The eluate of step b) is then applied to the column. Since very low salt concentrations may adversely affect the chromatographic separation properties of the column, the ionic strength is intended to be >20 mS/cm.

As an alternative to gel filtration, it is also possible to carry out a hydrophobic interaction chromatography (HIC). Hydrophobic interactions are of great biochemical importance. They are substantially involved in stabilizing three-dimensional tertiary structures of proteins. Hydrophobic interaction means the phenomenon of 2 hydrophobic molecules aggregating spontaneously in a polar environment (e.g. water). Dissolving a salt and increasing the ionic strength of the medium also increase the hydrophobic interaction of two non-polar molecules. Proteins have more or less high proportions of hydrophobic surface structures. They are therefore capable of adhering to hydrophobic adsorbents at an appropriately high ionic strength. The strength of the interaction may be controlled by the salt content and, in addition, by the choice of adsorbent. Examples of functional groups used are ethyl, butyl, propyl, octyl or else phenyl radicals. Adsorption is carried out in the presence of high salt concentrations; correspondingly, elution is carried out using a decreasing salt gradient. The salt used is normally ammonium sulphate.

In a particular embodiment of the invention, the composition or the eluate is filtered and/or concentrated prior to or after at least one of steps a) to c).

In order to concentrate proteins in solution, various processes may be used. This may be carried out, for example, by means of ultrafiltration. Here, a protein solution is pressed under pressure through a membrane through which only small molecules (salts, solvents) can pass, with larger molecules such as, for example, proteins being retained. This reduces the volume of the solution, thereby increasing the protein concentration. It is possible to use for filtration a tangential-flow filtration system, for example. Membranes suitable for this are the membranes which retain the protein. The filtration may be carried out by way of a pressure filtration in which the feed is pressed vertically against the membrane. As an alternative to this a cross-flow filtration process may be used in which the feed is directed tangentially across the membrane. The permeate passes through the membrane perpendicularly to the direction of flow. The continuous tangential skimming of the membrane achieves a purification effect which reduces the formation of a cover layer. For further improvement, it is also possible to arrange a fabric layer between two parallel membranes, which results in additional eddies.

In addition to concentration or alternatively thereto, the composition or the eluate may be filtered. Filter materials which may be used are cellulose nitrate, cellulose acetate, PVC, Teflon or ceramic membranes, for example made of zirconium oxide. The filters may be individual membranes or assembled in membrane systems, such as, for example, modules. The modules may be tubular modules, spiral modules or wound modules or hollow fibre modules.

In a preferred embodiment of the invention, the composition is clarified by filtration prior to step a) of the process for purification. The filters used for the clarifying filtration have a pore size of up to approximately 5 μm, preferably 4 μm, more preferably 3 μm and most preferably 1 μm.

The pore size of the filter used for ultrafiltration is preferably no more than 100 000 NMGT, more preferably no more than 75 000 NMGT, still more preferably no more than 50 000 NMGT and most preferably no more than 30 000 NMGT.

The pore size of the filters used for sterile filtration is preferably no more than 0.8 μm, more preferably no more than 0.6 μm, still more preferably no more than 0.4 μm and most preferably no more than 0.22 μm.

In a preferred embodiment of the invention, the pH of the eluate of step a) is made acidic prior to step b). An acidic pH is a pH of below 7.0, preferably below 5.5, more preferably below 4.0 and most preferably below 3.5. pH values below 3.5 are suitable for virus inactivation. After virus inactivation, the pH is raised again by adding a base, for example one tenth of the volume of 1 mol/l Tris pH 8.0. In addition to reducing the pH, the composition or any of the eluates may be filtered using a suitable filter, in order to remove viruses. Suitable filters are known to the skilled worker.

In a particularly preferred embodiment of the invention, the affinity chromatography column used in the process for purification is a protein-A chromatography column, the anion exchange chromatography column used in the process for purification is a Q-Sepharose column and the gel filtration column used in the process for purification is a Superdex-200 column.

Particular preference is furthermore given to a process for purification using the above-described steps a) to c), where appropriate incorporating the embodiments described, with the IL-15/Fc fusion protein, after purification, having a purity of at least 90%, preferably a purity of at least 95%, most preferably having a purity of at least 99%. The purity may be checked, for example, by HPLC-SEC, as described in the examples.

The present invention further relates to a purified IL-15/Fc fusion protein which has a purity of at least 90%, preferably a purity of at least 95%, most preferably a purity of at least 99%, it being possible for the purity to be checked, for example, by HPLC-SEC, as described in the examples.

In a further preferred embodiment, the average sialylation per N-glycan, i.e. the proportion of N-glycans occupied by sialic acid, in the purified IL-15/Fc fusion protein is at least 70%, more preferably at least 80%, still more preferably at least 90% and most preferably at least 95%.

In yet another preferred embodiment, the average sialylation per antenna, i.e. the proportion of antennae occupied by sialic acid, in the purified IL-15/Fc fusion protein is at least 50%, more preferably at least 70%, still more preferably at least 80% and most preferably at least 90%, the number of antennae corresponding to the number of branchings in N-glycan.

The average sialylation may likewise be determined by means of HPAEC-PAD (high performance anion exchange chromatography and pulsed amperometric detection), with the number of antennae being determined by means of HPLC. Sialic acid is the group name for N- and O-acylated neuraminic acid derivatives. Due to their exposed position as terminal group of a multiplicity of oligosaccharide chains of glycoconjugates, sialic acids make a considerable contribution to the biological properties of the former. Since enzymatic cleavage of sialic acid in glycoproteins results in a considerable shortening of their plasma half life, it is advantageous for sialylation to be as high as possible. This enables IL-15/Fc to be administered in lower doses and/or at longer intervals. The purification process according to the invention provides a gentle process which can be used to prepare an IL-15/Fc fusion protein which has an average sialylation of the values indicated above. N-Acetylneuraminic acid is the most frequently occurring sialic acid and an important component of glycoproteins. Like the other sialic acids, it protects against inactivation, and this is the reason for a high proportion of N-acetylneuraminic acid being advantageous.

In a further embodiment of the invention, the N-glycolylneuraminic acid portion of the sum of N-acetylneuraminic acids, N-glycolylneuraminic acids and asialo-N-glycans in the purified IL-15/Fc fusion protein is no more than 20%, preferably no more than 15%, more preferably no more than 10%. N-Glycolylneuraminic acid is a neuraminic acid which is usually not present in humans, which suggests that it may have antigenic action. Its proportion therefore should be as low as possible.

The amount of N-glycolylneuraminic acids as part of the sum of N-acetylneuraminic acids, N-glycolylneuraminic acids and asialo-N-glycans may be determined, for example, by means of HPAEC-PAD (high performance anion exchange chromatography and pulsed amperometric detection) by determining the percentage area of N-glycosylneuraminic acids of the total area of N-acetylneuraminic acids, N-glycolylneuraminic acids and asialo-N-glycans.

In principle, a large number of various biological functions of protein glycosylation are described. Thus it is possible for oligosaccharides to exert a protective or masking function by preventing recognition of the protein by proteases, microorganisms and antibodies. In many cases, glycosylation plays a structural and stabilizing part by assisting the correct folding of polypeptides in the rough endoplasmic reticulum and maintaining the conformation of a protein. Furthermore, glycosylation of a protein may modulate the interaction with ligands or receptors and play a part in cell-cell and cell-matrix recognition.

It is therefore desirable to provide an IL-15/Fc fusion protein which has as many of the naturally occurring or cell-produced glycosylations as possible. The number of glycosylation sites, the number of sugar molecules and/or the number of antennae may serve as a measure of this.

The present invention further relates to a composition comprising a purified IL-15/Fc protein and also excipients and additives.

Suitable excipients and additives which serve, for example, to stabilize proteins, are well known to the skilled worker (e.g. Sucker H. et al., (1991) Pharmazeutische Technologie, 2nd Edition, Georg Thieme Verlag, Stuttgart, Germany). They include, for example, physiological salines, Ringer dextrose, Ringer lactate, demineralized water, stabilizers, antioxidants, complexing agents, antimicrobial compounds, proteinase inhibitors and/or inert gases. In a preferred embodiment, the excipients and additives are mannitol, sucrose and/or glycine.

In a preferred embodiment, the composition has a pH of from 7.4 to 8.0, since liquid compositions containing IL-15/Fc and having this pH have proved to be particularly stable.

In a further preferred embodiment, the composition according to the invention is a pharmaceutical composition. A pharmaceutical composition is a composition which is intended and suitable to be used as medicament. It therefore comprises excipients and additives which are pharmaceutically suitable. Examples of those are Aqua sterilisata (sterilized water), substances influencing the pH, such as, for example, organic and inorganic acids and bases and salts thereof, buffer substances for adjusting the pH, isotonic agents such as, for example, sodium chloride, sodium bicarbonate, glucose and fructose, surfactants or surface-active substances and emulsifiers, such as, for example, partial fatty acid esters of polyoxyethylenesorbitan (Tween®) or, for example, fatty acid esters of polyoxyethylene (Cremophor®), fatty oils such as, for example, peanut oil, soybean oil and castor oil, synthetic fatty acid esters such as, for example, ethyl oleate, isopropyl myristate and neutral oil (Miglyol®), and also polymeric excipients such as, for example, gelatine, dextran, polyvinylpyrrolidone, solubility-increasing additives of organic solvents such as, for example, propylene glycol, ethanol, N,N-dimethylacetamide, propylene glycol or complexing agents such as, for example, citrates and urea, preservatives such as, for example, hydroxypropyl and methyl benzoate, benzyl alcohol, antioxidants such as, for example, sodium sulphite, and stabilizers such as, for example, EDTA, PVP, cellulose esters as granulating or release-slowing agents, such as, for example, waxlike and/or polymeric substances based on Eudragit®, cellulose or Cremophor®, antioxidants, sweeteners such as, for example, sucrose, xylitol or mannitol, flavourings, aromatizers, preservatives, colorants, buffer substances, direct tabletting agents, such as, for example, microcrystalline cellulose, starch and starch hydrolysates (e.g. Celutab®), lactose, polyethylene glycols, polyvinylpyrrolidone and dicalcium phosphate, lubricants, fillers, such as, for example, lactose or starch, binders in the form of lactose, types of starch, such as, for example, wheat or corn or rice starch, cellulose derivatives such as, for example, methylcellulose, hydroxypropylcellulose or siliceous earth, talcum, stearates such as, for example, magnesium stearate, calcium stearate, talc, siliconized talc, stearic acid, cetyl alcohol or hydrogenated fats.

The invention will be illustrated below by examples and figures which are not to be construed as being limiting.

### DESCRIPTION OF THE FIGURES

FIG. 1 depicts a map of the pcDNA3.1hCD5.6Ala7 expression construct.

FIG. 2-3 depicts the sequence of the pcDNA3.1hCD5.6Ala7 expression construct (SEQ ID NO. 1).

FIG. 4 depicts a map of the pMG10Ala7 expression construct.

FIG. 5-6 depicts the sequence of the pMG10Ala7 expression construct (SEQ ID NO. 2).

FIG. 7A depicts the nucleic acid sequence of the human mutated IL-15/Fc with CD5 leader (SEQ ID NO. 3).

FIG. 7B depicts the amino acid sequence of the human mutated IL-15/Fc with CD5 leader (SEQ ID NO. 4).

FIG. 7C depicts the amino acid sequence of the murine mutated IL-15/Fc with CD5 leader (SEQ ID NO. 5).

FIG. 8 depicts the IL-15/Fc content in cell culture supernatants of CHO-K1 cells after transfection with the pcDNA3.1hCD5.6Ala7 plasmid which contained the leader sequence indicated in each case.

FIG. 9 depicts the IL-15/Fc content in cell culture supernatants of CHO-K1 cells after transfection with various expression constructs. Each bar represents the average+SEM of duplicate determinations of in each case 2 independent experiments.
pcDNA3.1 corresponds to the pcDNA3.1hCD5.6Ala7 vector.
pVS8-Ala7 corresponds to pSwitch plasmid (Valentis) with the construct for IL-15/Fc construct.
pMG-Ala7 corresponds to pMG plasmid (Invivogen) with the construct for IL-15/Fc construct.
pCINeo-Ala7 corresponds to pCI-Neo plasmid (Promega) with the construct for IL-15/Fc construct.

### EXAMPLES

### Example 1

### Production of IL-15/Fc in CHO-K1 Cells

To produce a CHO-K1 producer cell line for IL-15/Fc, an expression construct for IL-15/Fc should be formed and optimized with regard to its secretory properties, to the identity/integrity of the fragments which it contains and to suitable resistance genes.

### a) Starting Materials

A human IL-15/Fc expression construct (mutated IL-15/human Fc) was provided by the Department of Immunology of the “Beth Israel Deaconness Medical Center” (Harvard Medical School, Boston, USA).

The oligonucleotides were obtained from MWG-Biotech (Ebersberg, Germany). The sequences of the relevant signal peptides were obtained from gene libraries.

The restriction enzymes (BgIII, XBaI, BamHI, SmaI, BstXI, ApaI), Lipfectamin2000, other molecular-biological reagents (T4-DNA ligase, T4-polynucleotide kinase) and the plasmids pSecTagA, pcDNA3.1 were obtained from Invitrogen (Karlsruhe, Germany) or Amersham-Pharmacia (NheI, Protein A Sepharose Uppsala, Sweden).

Competent E. coli XL10-Gold cells were obtained from Stratagene (La Jolla, USA). The BCA kit (Pierce) was purchased from KMF Laborchemie (Sankt Augustin, Germany).

The plasmid-DNA purification kits (Endofree-Maxi kit, Endofree-Giga kit) were from Qiagen (Hilden, Germany).

The antibodies were obtained from BD-Pharmingen (mouse-anti-h IL-15; catalogue number 554712; Heidelberg, Germany) and Dianova (goat-anti-mouse-POD; catalogue number 15-036-003; goat-anti-human-POD; catalogue number 109-036-088; Hamburg, Germany).

### b) Methods/Results

The starting plasmid contained within the pSecTagA vector backbone the cDNA of a fusion protein comprising a mutated human IL-15 fused to the Fc part (hinge region and CH2, CH3 region) of human IgG1. The structure of the plasmid corresponds to that described by Kim at al. (J. Immunol., 160: 5742-5748; 1998), except that the Fc part cited in this publication is a murine Igγ2a.

The Igk leader which is already present in the pSecTagA vector was used for secretion of the fusion protein by in-frame cloning of the IL-15/Fc part. For this, the intrinsic signal sequence was removed from the native IL-15 sequence. Due to the cloning, however, 10 additional amino acids were introduced between the 3′ end of the Igk leader sequence and the 5′ end of the IL-15 coding sequence, which were retained in the secreted protein after processing of the protein. In order to remove these unspecific amino acids and to improve the secretory properties of the protein, various leader sequences of other secretory or cell-surface proteins were tested: murine Igk (Coloma et al., J. Immun. Methods 152: 89-104; 1992; accession number X91670), human CD5 (Jones et al., Nature 323: 346-349; 1986; accession number X04391), CD4 (Hodge et al. Hum. Immunol. 30: 99-104; 1991; accession number M35160), MCP-1 (Yoshimura et al. Je. FEBS Lett. 244: 487-493; 1989; accession number M24545) and IL-2 (Taniguchi et al., Nature 302: 305-310; 1983; accession number K02056), (accession numbers are based on the National Center for Biotechnology Information). After removing the Igk leader and the additional amino acids, the leader was replaced with the signal peptide sequences mentioned by cloning double-stranded oligonucleotides. The identity was checked by sequencing. Subsequently, the resulting constructs were tested by transient transfection of HEK-293 cells, using Lipfectamin2000. The protein content of the cell culture supernatants of the cells which have been transfected with the various constructs was measured by means of the BCA assay, after a protein-A-Sepharose purification according to the method by Moll and Vestweber (Methods in Molecular Biology, 96: 77-84, 1999). The identity of the protein was checked by means of silver staining of the SDS gel and Western blots against either the Fc or the IL-15 part, in order to ensure the presence of both components of the fusion protein. The best results were obtained in the experiments described when using the CD5 leader. The latter was selected for further vector optimization, with the leader itself no longer being present in the secreted fusion protein.

It was furthermore tested, whether replacing the cDNA of the Fc part with the genomic DNA containing Exon/Intron structures also contributes to improved protein expression. The presence of Introns which have to be removed by the splice apparatus of the nucleus may improve RNA export from the nucleus and also RNA stability. Therefore, the genomic Fc part was bound to the IL-15 cDNA sequence by inserting splice-donor and—acceptor sites. The resulting plasmids were likewise modified by various leader sequences and tested as described above. Protein analysis by Western blot, however, revealed that various undesired splice variants were present so that it was decided to continue using the cDNA form of the Fc part.

Consequently, the resulting plasmid comprises a human CD5 leader and a cDNA Fc part.

Sequencing of the mutated IL-15/Fc expression construct revealed that the Fc part contained 3 mutations which were already present in the original construct. Two of these mutations related to amino acids at highly conserved positions. The third mutation was a Cys-Ala mutation at position 4 in the hinge region which was inserted deliberately in order to stop the formation of intra- and intermolecular cysteine bridges.

In order to remove the two undesired mutations while retaining the Cys-Ala mutation, the Fc cDNA was subcloned by means of RT-PCR. The RNA source used was a CHO-K1 cell line transfected with a construct coding for a VCAM-1-Fc fusion protein. The amplified Fc cDNA fragment was cloned into the CD5-mutIL-15 plasmid, and the Fc part was removed by BamHI/XbaI restriction.

The resulting plasmid was analyzed again on the basis of distinct restriction patterns and by means of subsequent sequencing and referred to as CD5-6Ala7. Since the use of Zeocin as DNA-intercalating agent could cause mutations, the expression cassette for IL-15/Fc was removed from the original pSecTagA backbone and cloned into pcDNA3.1 which contains the neomycin resistance gene under the control of the SV40 promoter. Both strands of the resulting plasmid were sequenced and revealed complete correspondence with the IL-15/Fc expression cassette.

The construct was again tested for its protein expression by means of transient transfection of CHO-K1 cells and Western blot analyses of the cell culture supernatant. As a positive control, a transfection with the CD5-6A1a7 plasmid was carried out in a parallel experiment.

To this end, on the day before transfection the cells were seeded in triplicates at a density of 5×10⁵ cells per well in tissue culture plates with 6 wells. 2 μg of plasmid and 4 μl of Lipofectamin2000, each of which were diluted in 250 μl of Optimem1 medium, were used for transfection. Both solutions were mixed and, after incubation at room temperature for 30 min, the mixture was pipetted into the culture media of the tissue culture plates.

2 days after transfection, the culture medium was removed and analyzed for its IL-15/Fc content by means of a Western blot against the human IL-15 part: 20 μl of the cell culture supernatant were mixed with 5 μl of 5×Laemmli buffer and incubated at 85° C. for 5 min. The samples were then run across a 12% polyacrylamide gel.

The gel was then blotted using a semi-dry blotting chamber. The blot was treated with blocking solution containing 5% milk powder in PBS, 0.1% Tween20 overnight. The blot was then incubated with a monoclonal mouse-anti-human-IL-15 antibody in a 1:1000 dilution in blocking solution for 4 hours. After 3 washing steps (10 min PBS, 0.1% Tween20), the blot was incubated with the secondary antibody goat-anti-mouse peroxidase (dilution 1:5000) at room temperature for another 2 hours. The blot was washed again 3 times and then Lumilight solution was applied dropwise to the blot surface and an X-ray film was exposed to the blot.

Specific Western blot signals within the range of signals obtained after transfection with CD5-6Ala7 revealed that the cell culture supernatants of all 3 parallel transfections contained IL-15/Fc as protein.

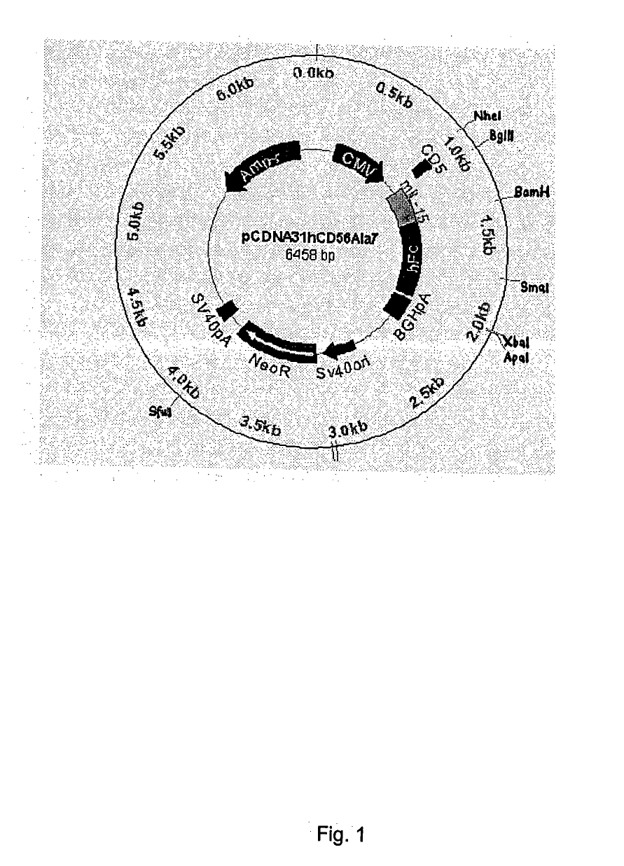

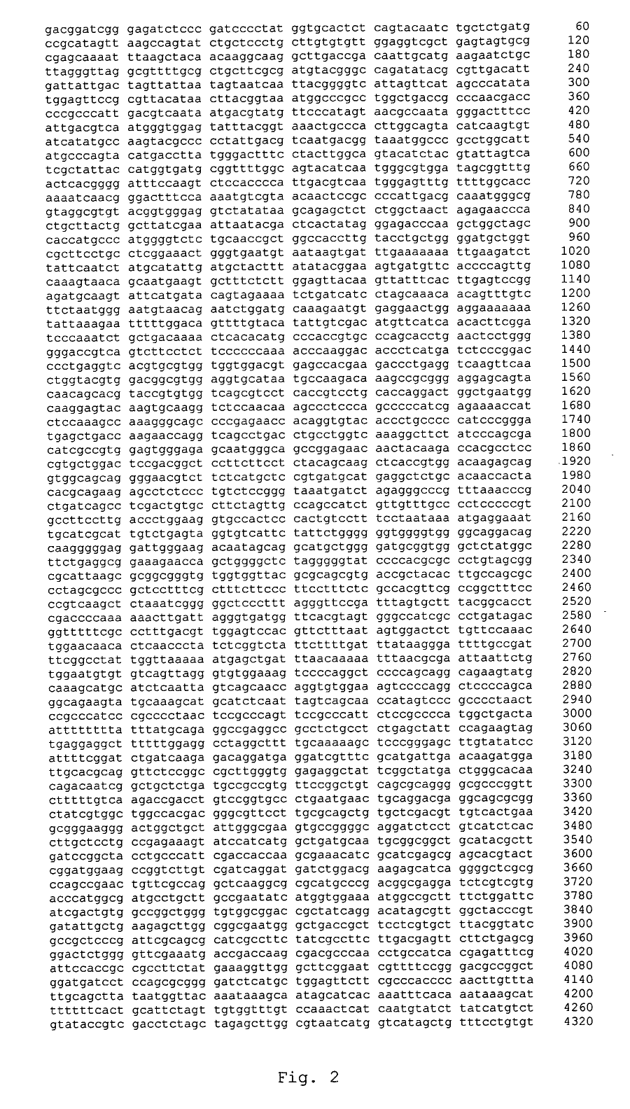

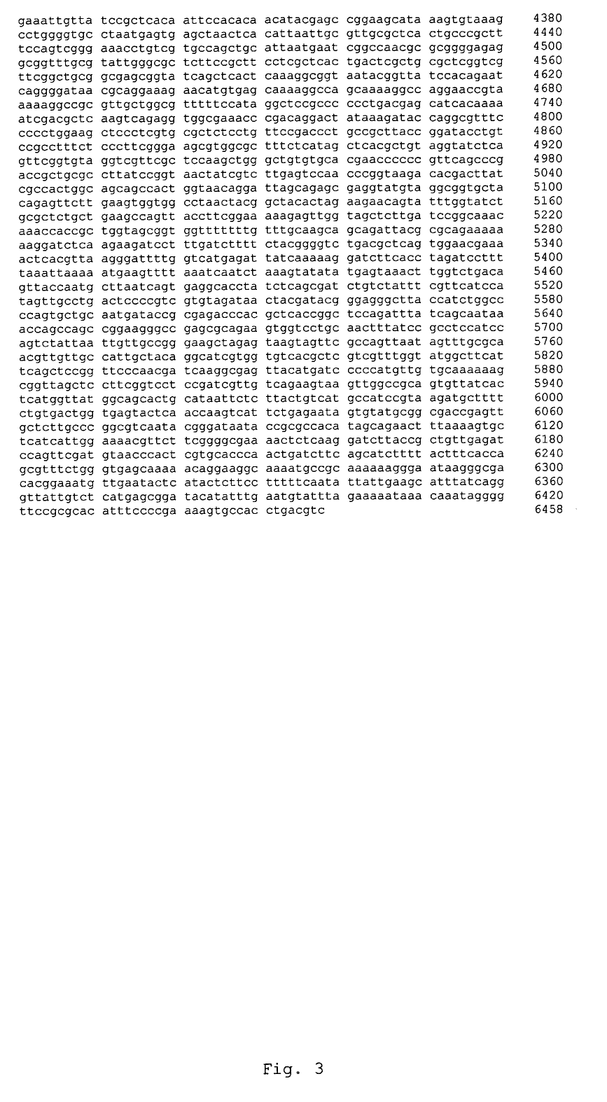

It was therefore shown that the pcDNA3.1hCD5.6Ala7 plasmid (FIGS. 1 to 3) can be used for protein expression in CHO-K1 cells.

### c) Conclusions

An IL-15/Fc plasmid, pcDNA3.1hCD5.6Ala7, was prepared, which contained an expression cassette containing a CD5 leader with a mutated human IL-15 fused to the cDNA of human IgG1-Fc under the control of the CMV promoter. To select stable eukaryotic cell clones, a neomycin resistance gene was introduced. The plasmid was sequenced and revealed 100% correspondence in the relevant coding regions, with only a slight discrepancy (repeat of 3 base pairs) without any relevance in the vector backbone. The functionality of the construct was checked by transient transfection of CHO-K1 cells.

### Example 2

Transfection of eukaryotic cell lines (e.g. CHO-K1 cells) with a plasmid containing the DNA of the desired product is a standard process for producing therapeutic proteins. Nevertheless, the low productivity levels of the stable cell clones produced in this way are a widely known problem. There are therefore various strategies to increase the productivity of an existing cell line. Apart from the attempt to increase the number of plasmid copies in the cell (e.g. via the Methotrexat/DHFR system), it is furthermore possible to modify the expression construct itself. In addition to a strong promoter (e.g. the CMV promoter), introducing an Intron possibly results in better RNA stability and better RNA export from the nucleus, which is carried out by the splice apparatus of the cell. Nevertheless, a test must be carried out as to which combination of Intron/transgene is suitable. For this purpose, various Introns were combined with the human IL-15-Fc in order to find a combination which increases IL-15-Fc production by CHO-K1 cells.

### a) Materials

The plasmid used as starting plasmid was the pcDNA3.1hCD5.6Ala7 plasmid. It is depicted schematically in FIG. 1. Its sequence is disclosed as SEQ ID NO. 1.

The test system used was either CHO-K1 cells (DSM, Brunswick, Germany, accession number ACC110) or HEK-293 cells (Qbiogene, Grünberg, Germany, AE80503, QBI-293A). E. coli cells (XL10-Gold, Strategene, La Jolla, USA) were also used. The cells were cultured under standard culturing conditions (5% CO₂, 37° C., humidified atmosphere). The CHO-K1 cells were passaged twice a week at a ratio of 1:20, with the HEK-293 cells being passaged at a ratio of 1:6. The medium used was DMEM-F12+10% FKS+1% PEN/Strep for the CHO-K1 cells and DMEM+Glutamax+10% FKS+1% PEN/Strep for the HEK-293 cells. Optimem1 medium was used for transfection. All media were used by Invitrogen, Karlsruhe, Germany (catalogue numbers 31331-028; 32430-027; 51985-018). The plasmid used was pCl-Neo (Promega) containing a CMV promoter and a chimeric Intron, a 5′ splice-donor site of the human beta-globin gene and a 3′ splice-acceptor site of the IgG heavy chain of the variable region. pMG (Invivogen) is a prolonged CMV promoter containing an IntronA from CMV. pSwitch (Valentis) is a synthetic Intron, IVS8. Furthermore, the following enzymes and restriction enzymes were used: ApaI, EcoRV, XbaI, NruI, PacI, SmaI, XhoI, T4-DNA ligase, T4-DNA polymerase, alkaline phosphatase from calf intestine. These and other molecular-biological reagents (Lipofectamin2000) were obtained from Invitrogen. NheI was obtained from Amersham-Pharmacia (Uppsala, Sweden) and the plasmid purification kits were obtained from Qiagen, Hilden, Germany. The “Expand High Fidelity PCR System” (catalogue number 1 732 641) was obtained from Roche, Mannheim, Germany.

### b) Methods

i) The IL-15/Fc insert of the pcDNA3.1hCD5.6Ala7 plasmid was isolated by NheI/ApaI digestion. The plasmid was first linearized by ApaI restriction and the 5′-protruding ends were blunted by means of T4-polymerase treatment. The IL-15/Fc insert was then isolated by way of a subsequent NheI digestion. The fragment was ligated with pcI Neo which had been digested with NheI and SmaI.
ii) The CMV promoter of pcDNA3.1hCD5.6Ala7 was removed and replaced with the extended CMV promoter with intron A, derived from pMG: the pMG plasmid was cleaved with PacI and the protruding ends were blunted by means of T4-polymerase treatment. After a second XbaI treatment, the 1.7 kb fragment obtained in this way, which contained the CMV promoter+intron A, was purified by agarose gel electrophoresis. The CMV promoter was removed from pcDNA3.hCD5.6Ala7 by means of NheI and subsequent NruI restriction digestions. The resulting fragment was ligated with the pMG-promoter intron at 4° C. overnight.
iii) The IVS8 intron was amplified by means of PCR and cloned between the 3′ end of the CMV promoter and the 5 end of the IL-15 insert in pcDNA3.1hCD5.6Ala7. The plasmid was linearized by means of NheI restriction digestion and subsequently treated with alkaline phosphatase from calf intestine. The intron was amplified by means of PCR using primers which contained XbaI restriction cleavage sites and using the Expand High Fidelity PCR system under the following conditions: the reaction mixture used consisted of 2 μl of dNTPs (Qiagen, Taq core kit, 2 mmol/l each), 25 μmol of primers, 5 μl of 10× buffer, 0.75 μl of High Fidelity Taq Polymerase, 1 μl (approximately 15 ng) of pSwitch-XhoI/EcoRV fragment, and water to give a volume of 50 μl. The PCR program (25 cycles) was as follows: 5 min at 95° C., 15 s at 94° C., 30 s at 55° C., 30 s at 72° C., 5 min at 72° C. The PCR product was cleaved with XbaI, eluted from a 0.8% agarose gel and ligated with the linearized plasmid.

The resulting plasmids were transformed into E. coli XL10 Gold and the plasmids were analyzed by means of Miniprep. One clone each of any plasmids exhibiting a suitable restriction pattern was used for the subsequent endotoxin-free plasmid preparation.

IL-15/Fc expression was analyzed after transient transfection of HEK-293 or CHO-K1 cells. On the day before transfection, the cells were seeded at a density of 5×10⁵ cells per well in cell culture plates with six wells in duplicates. Transfection according to Felgner et al. (Proc. Natl. Acad. Sci. USA, 84:7413-7417; 1987) was carried out by diluting 2 μg of plasmid and 4 μl of Lipofectamin2000 in each case in 250 μl of Optimem1 medium. Both solutions were mixed and, after incubation at room temperature for 30 minutes, the mixture was pipetted into the cell culture medium in the cell culture plates. Two days after transfection, the culture medium was removed and the IL-15/Fc content was determined using an ELISA test directed at the Fc part of IL-15/Fc.

### c) Results

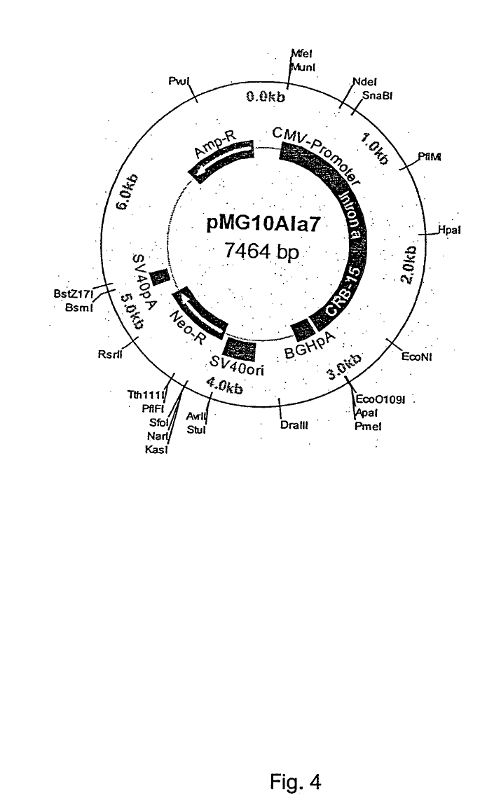

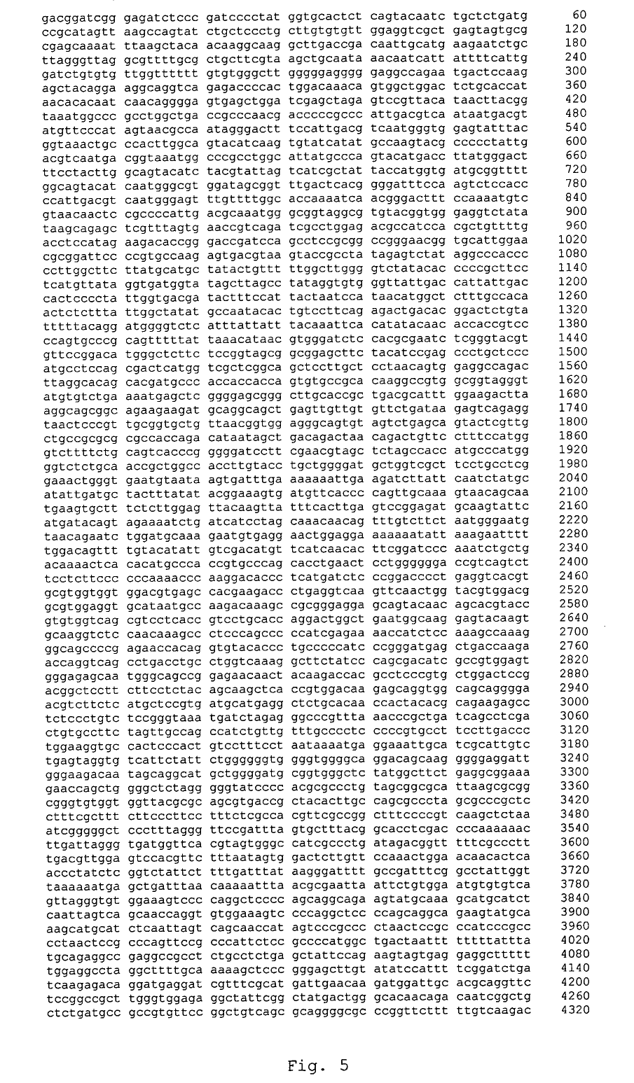

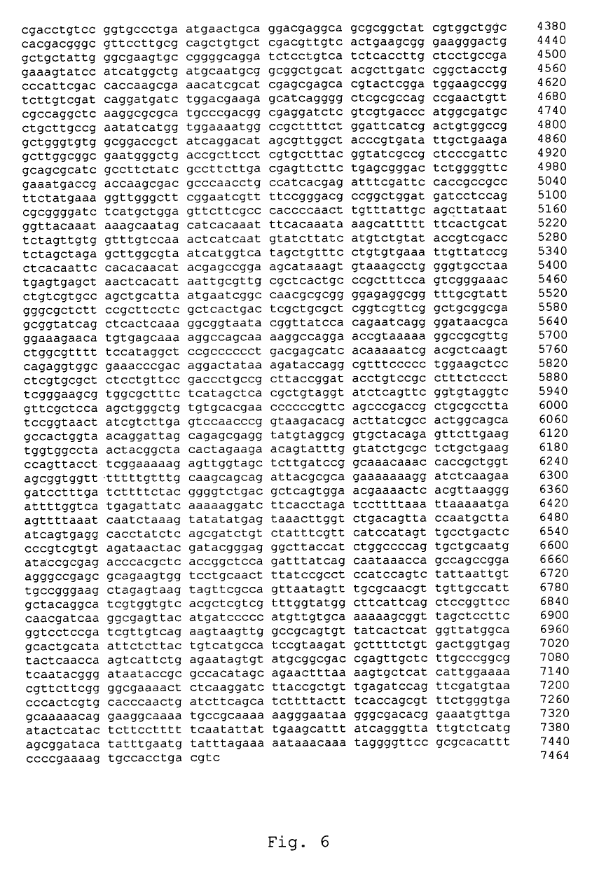

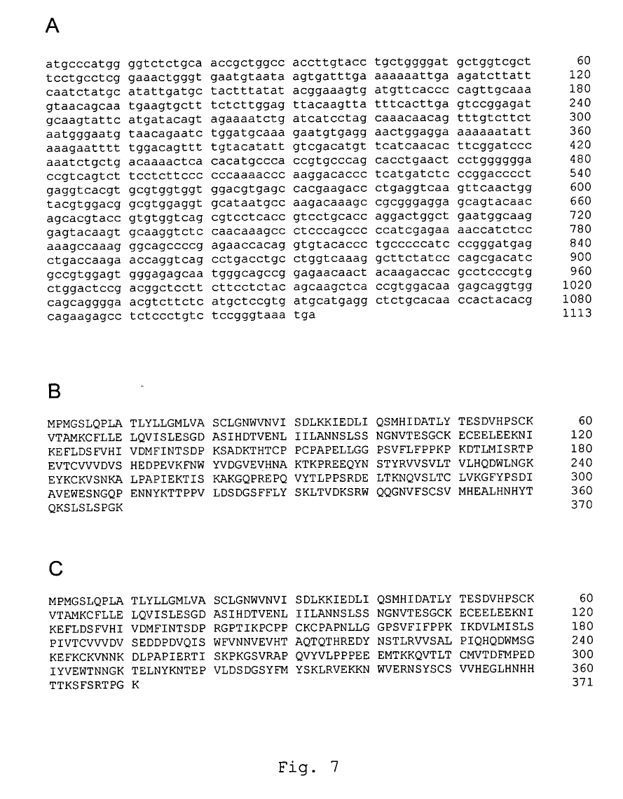

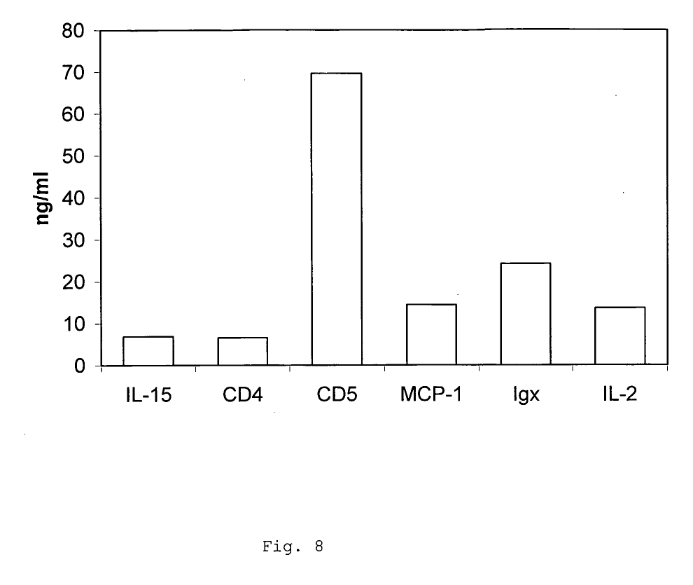

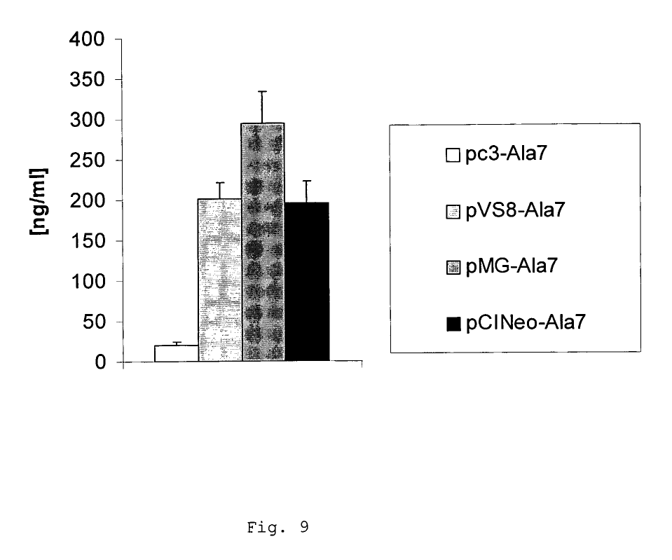

IL-15/Fc secretion by HEK-293 cells transfected with various expression constructs was barely influenced by other vector components. In contrast, IL-15/Fc expression by CHO-K1 cells increased by a factor of 200-300, after an intron had been inserted into the IL-15/Fc construct. The original construct, pcDNA3.1hCD5.6Ala7, resulted in levels of protein secretion which were hardly detectable (below 10 ng/ml), with insertion of an intron, after transfection of the cells with pMG10Ala7 (FIGS. 4 to 6; SEQ ID NO. 2), resulting in IL-15/Fc levels of approximately 300 ng/ml. The ELISA data which indicate the IL-15/Fc-expression levels in CHO-K1 çells are depicted in FIG. 4. Since the expression levels were highest after transfection with the pMG construct, the latter was selected for producing a stable CHO-K1-expression cell line.

To this end, the plasmid was first subjected to single-strand sequencing. Both strands of the construct were sequenced in the region which contained the IL-15/Fc cassette, the newly inserted CMV promoter and the intron fragment. The plasmid contained the IL-15/Fc cassette under the control of the CMV promoter. The intron A which derived from CMV (MG plasmid) was positioned between the promoter and the translation initiation site. The plasmid contained a BGHpolyA site downstream of the IL-15/Fc fragment; the neomycin-resistance gene was controlled by an SV40 promoter and also contained an SV40polyA site. The plasmid contained an ampicillin-resistance gene for selection and amplification in E. coli.

### d) Discussion and Conclusions

In order to increase the protein yield of stable CHO-K1-IL-15/Fc transfectants, the expression plasmid was modified by introducing an intron between the promoter and the IL-15/Fc cassette. The combination intron-transgene-host cell strongly affects protein expression, and it is therefore not possible to predict which intron is the most effective in increasing IL-15/Fc expression in the two cell types analyzed.

While HEK-293 cells were barely influenced by introduction of the intron, a large increase in IL-15/Fc secretion was detected in CHO-K1 cells. Expression of the IL-15/Fc protein in CHO-K1 cells increased by much more than an order of magnitude in comparison with the original IL-15/Fc expression vector, with a plasmid being used which contained the CMV promoter and intron A from pMG. The plasmid may be used for producing an IL-15/Fc producer cell line which may be used for producing IL-15/Fc for pre-clinical and clinical studies or else for industrial IL-15/Fc production.

### Example 3

### Purification of the IL-15/Fc fusion protein

### a) Clarification and Concentration

Approximately 3100 litres of the supernatant from Example 2 (pMG10Ala7 plasmid) were clarified, concentrated and sterile-filtered in 6 runs. This involved clarifying the supernatant by means of Profile Star filters (3 μm, 20 inches, Pall Corporation, East Hills, N.Y., USA). Subsequently, the supernatant was concentrated 10 to 15 times by means of a tangential-flow filtration system using 2.0 m² of Biomax-30 membrane (Millipore, Billerica, Mass., USA) in total. The inlet pressure was 2-2.5 bar, the outlet pressure was 1.5 bar. After the concentration, the concentrates were sterile-filtered through a filter system consisting of a prefilter (Polysep II (0.2 μm, 10 inch, Millipore, Billerica, Mass., USA)) and a final filter (Durapore (0.22 μm, 10 inch, Millipore, Billerica, Mass., USA)). Concentration of the six different runs took between 4.5 and 8.5 hours.

b) rProtein-A Chromatography

Approximately 240 litres of the pooled yield of the clarification and concentration steps were applied to an rProtein-A column (2.6 litres). The flow rate during loading was between 10 and 15.4 l/h (65-100 cm/h). Subsequently, the column was washed with 10 column volumes of 20 mmol/l Tris, 150 mmol/l NaCl, pH 7.5, followed by 10 column volumes of 0.1 mol/l citrate, pH 5.0. IL-15/Fc eluted at 5 column volumes of 0.1 mol/l citrate, pH 3.4, and the column was stripped with 5 column volumes of 0.1 mol/l citrate, pH 3.0. IL-15/Fc eluted in a single sharp peak. The eluate was yellowish and contained particulate components. The eluate was immediately adjusted to pH 3.5 with 1 mol/l citrate. Virus inactivation at low pH was carried out with continuous stirring at room temperature for 1 hour. The treatment was stopped by adding 1 mol/l Tris and adjusting the pH to 8.0. The eluate (8.6 litres) was sterile-filtered using Millipak-20 filters and stored in 10-litre Schott bottles at 2-8° C.

### c) Q-Sepharose Chromatography

A Q-Sepharose-FF column was packed to a height of 12.7 cm, corresponding to a column volume of 1.9 litres. After equilibration, the HETP (Height Equivalent to a Theoretical Plate) was 0.041 and the asymmetry was 1.1.

The eluate of the rProtein-A chromatography was diluted 1:5 with 20 mmol/l Tris, pH 8.0, in a 50-litre Stedim bag. 42.2 kg of diluted rProtein-A eluate were applied to the 1.9-litre Q-Sepharose column. The column was washed with 5 column volumes of 20 mmol/l Tris, 0.1 mol/l NaCl, pH 8.0, followed by 5 column volumes of 20 mmol/l Tris, 0.2 mol/l NaCl, pH 8.0. IL-15/Fc eluted with 5 column volumes of 20 mmol/l Tris, 0.35 mol/l NaCl, pH 8.0. The column was stripped with 5 column volumes of 20 mmol/l Tris, 1.0 mol/l NaCl, pH 8.0. The eluate of the Q-Sepharose chromatography (5.0 litres) was sterile-filtered into a 10-litre Schott bottle, using a Millipak-20 filter, and stored at 2-8° C. The eluate was still slightly yellowish, although a large part of the yellow contamination had been removed.

Monitoring the optical density at 280 nm revealed that IL-15/Fc eluted in a single sharp peak towards the end of elution.

d) Concentration of the Eluate from Q-Sepharose Chromatography by Ultrafiltration

The Q-Sepharose chromatography eluate was concentrated to a final concentration of 8.85 g/l, based on OD₂₈₀ (optical density measured at 280 nm). This involved the use of a 0.1 m² Biomax 10-kD membrane. The inlet pressure was adjusted to 1.0 bar and the outlet pressure to 0.5 bar. The concentrate (1.9 litres) was sterile-filtered in 5 aliquots into 0.5-litre PETG bottles.

### e) Superdex-200 Chromatography

The columns were filled with the column material under a pressure of 3 bar. The column used had an HETP of 0.013 cm and an asymmetry of 3.0.

The 5 aliquots of the concentrates were purified by 5 successive runs via the 13.5-litre Superdex-200 column. The chromatograms depict a small peak at high molecular weights and a main peak at monomeric IL-15/Fc. The fractions were collected, resulting in the following fractions.

| Fraction | collected at a CV of |
|---|---|
| 1 | 0.32-0.41 |
| 2 | 0.41-0.42 |
| 3 | 0.42-0.43 |
| 4 | 0.43-0.44 |
| 5 | 0.44-0.45 |
| 6 | 0.45-0.46 |
| 7 | 0.46-0.48 |
| 8 | 0.48-0.50 |
| 9 | 0.50-0.52 |
| 10 | 0.52-0.54 |
| 11 | 0.54-0.56 |
| 12 | 0.56-0.58 |
| 13 | 0.58-0.60 |

CV: Column volume

### f) Pooling of Superdex Fractions

The fractions from the Superdex-200 purifications were used for the further preparation process based on their purity determined in HP-SEC analyses. Usually, fractions 5 to 11 were pooled for this purpose. The amount of IL-15/Fc was calculated on the basis of the peak areas of the HP-SEC analyses.

### g) Virus Filtration

The pooled Superdex fractions of all 5 runs were transferred to a Stedim bag, pooled and diluted with PBS (pH 7.4) to a final IL-15/Fc concentration of 0.88 g/l. The diluted, pooled Superdex fractions were filtered successively through a 1-m² Planova-35N and a 1-m² Planova-15N filter. The working pressure was 0.5 bar in the case of the Planova-35N filter. After the filtration, the filters were rinsed with 3 l of PBS (pH 7.4). The flow through the virus filter was between 12 and 15 litres per hour during loading at 0.5 bar. During washing, flow decreased to approximately 10 litres per hour, since washing was carried out at 0.4 bar.

### h) Sterile Filtration

16.4 kg of final products were sterile-filtered using a 0.22 μm Millipak filter and introduced into 1-litre PETG bottles. The bottles were stored at 2-8° C. until further use.

### Example 4

### Specification of the IL-15/Fc Fusion Protein

### a) SDS-PAGE/Western-Blot Analyses

Analyses on reduced SDS-PAGE with subsequent silver staining indicated that IL-15/Fc in the Q-eluate, in the pooled Superdex fractions and in the final product had a migration behaviour similar to IL-15/Fc of the control. In a few gels, additional bands at lower and higher molecular weights were visible in the Q-eluate, which then, however, were also present in the control. Two bands with higher molecular weights were visible in a few of the pooled Superdex fractions. These bands were then also detected in the control. The additional bands with high molecular weights are probably due to an insufficient reduction of the samples prior to loading the gel, probably caused by longer standing times.

Besides the main IL-15/Fc band, no additional bands were detectable in the case of the IL-15/Fc final product on a non-reduced SDS-PAGE with subsequent silver nitrate staining. The non-reduced Western blot with subsequent detection of Fc also showed only the IL-15/Fc main band. This proves the fact that the quality of IL-15/Fc is comparable during all process steps.

### b) N-Glycosylation of IL-15/Fc

The N-glycosylation properties of IL-15/Fc in the crude extract, the clarification and purification steps, of the purification intermediates and of the final product were determined. The average sialylation per native N-glycan and the percentage area of N-acetylneuraminic acid in relation to the sum of the areas of N-acetylneuraminic acid, N-glycosylneuraminic acid and the asialo-N-glycans were determined by means of HPAEC-PAD analysis (HPLC: BioLC, detector PAD, column: CarboPacPA 100 Analytical (2×250 mm), buffer A: 0.1 mol/l NaOH; buffer B: 0.1 mol/l NaOH, 0.6 mol/l sodium acetate, for determining native N-glycans, or buffer A: 0.2 mol/l NaOH; buffer B: 0.2 mol/l NaOH, 0.6 mol/l sodium acetate, for determining asialo-N-glycans). The results are summarized in the table below:

| | Average sialylation | % N-acetyl- |
|---|---|---|
| Purification step | per N-glycan | neuraminic acid |
| Harvest | 2.16 | 60 |
| Clarification and | 2.17 | 61 |
| concentration | | |
| rProtein-A eluate | 1.90 | 48 |
| Q-Sepharose eluate | 2.01 | 50 |
| Concentrate | 1.95 | 49 |
| Superdex 200 | 2.04 | 51 |
| Final product | 2.00 | 44 |

A decrease in the average sialylation and the percentage of N-acetylneuraminic acid was observed between clarification and concentration and rProtein-A chromatography, when the samples were left standing for a longer time. The glycosylation of all other purification steps is comparable.

### c) Host-Cell Proteins

The test for host-cell proteins was carried out at NewLab AG on two different days, using a conventional ILA test. This resulted in finding 2.1×10³ and 3.9×10³ ng/ml host-cell protein, corresponding to 2.9×10³ ppm and 5.3×10³ ppm, respectively. An additional test for host-cell proteins in the final product, using the Cygnus test (article No. F015), gave 240 ng/ml foreign protein, corresponding to 3.3×10² ppm. At shorter standing times, lower values were also measured.

### d) N-Terminal Sequence

The terminal IL-15/Fc sequence was determined in the final product. This test revealed an N-terminal sequence of (Asn/Asp)₁-Trp₂-Val₃-Asn₄-Val₅-Ile₆-Ser₇-Asp₈-Leu₉-Lys₁₀. The amino acid sequence determined in this test corresponds to the expected N-terminal IL-15/Fc sequence, except that N-terminal ASN is deamidated by approximately 10% (sometimes also higher values, but no more than 20%), a standard deamidation of 10%, due to the Edman process, having been taken into account. Longer standing times resulted in a deamination of approximately 45%.

### e) Potency

The potency of IL-15/Fc in the final product was determined by means of a bioactivity test. This test detects the bioactivity of the IL-15 antagonist IL-15/Fc by studying the proliferation-inhibiting action on IL-15-stimulated murine CTLL-2 cells as a function of the dose. The ED₅₀ was 1.65 ng/well.

### f) Protein-A Remnants

rProtein-A which had been washed out of the rProtein-A column was determined in the final product by means of an rProtein-A-specific ELISA. This remnant was found here to be <0.001%.

### g) DNA

The content of residual DNA in the final product was determined by means of Q-One. Less than 5 pg/0.5 ml residual DNA were found, correspondingly less than 14 pg of residual DNA per mg of IL-15/Fc.

### h) Geneticin

Geneticin was added during the preculture of the fermentation process. It was therefore investigated, whether geneticin had been removed sufficiently. The geneticin concentration was determined to be less than 30 ng/ml in the IL-15/Fc final product.

### i) Specifications

The IL-15/Fc product obtained by means of the process described above had the following specifications:

| | |
|---|---|
| Product concentration | 0.70 mg/ml |
| Product purity | 99% |
| pH | 7.4 |
| ED₅₀ | <0.1 μg/well |

The product identity was confirmed by means of detecting both the IL-15 part and the Fc part. The bands in reduced and non-reduced SDS-PAGE also corresponded to the control bands.

### Example 5

### Preparation of Formulations

### a) Influence of the pH

IL-15/Fc was diluted with various buffer substances and additives to 1 mg/ml, as indicated in the table. After their preparation, the buffers had been filtered here using a 0.2-μm filter (Acrodisc Syringe Filter, Pall). The finished preparations were stored in 10-ml glass vessels (4 ml/vessel) at 40° C. and 75% relative humidity.

| Formulation | Buffer | PBS |
|---|---|---|
| 1 | 35.7 mmol/l citrate pH 4 | 2.73 mmol/l |
| 2 | 35.7 mmol/l acetate pH 5.5 | 2.73 mmol/l |
| 3 | 35.7 mmol/l citrate pH 6 | 2.73 mmol/l |
| 4 | 12.6 mmol/l PBS pH 7.4 | |
| 5 | 35.7 mmol/l Tris pH 8 | 2.73 mmol/l |

The purity of the IL-15/Fc formulations was determined by means of HP-SEC. A TSK-GEL G3000SWXL column (300×7.8 mm) and, as a guard column, a TSK-GELSWXL column (40×6 mm) were used for this purpose. They were operated at 25° C. and a flow rate of 1 ml per minute. The injection volume was 50 μl. Detection was carried out by means of a detector at 214 nm. At time 0, the purity was only 85%, since the IL-15/Fc sample used contained approximately 15% aggregation products. Therefore, 85% of the starting material are in the active-dimer form. At pH 4, the peak area of the IL-15/Fc dimer decreased from approximately 85% to approximately 40% after 7 days. A decrease in purity was also observed at pH 5.5 and pH 6. The formulations at pH 8 and pH 7.4 proved to be stable at 40° C. over the period of 7 days. This suggests a decrease in the stability of IL-15/Fc with decreasing pH.

The formulations were furthermore subjected to a non-reduced SDS-PAGE at 40° C. after 7-day storage. The pH 4 formulation contained two bands with degradation products at approximately 70 and 50 kD. The pH 6 and pH 5.5 formulations also contained two bands with degradation products, but these bands had an apparent molecular weight of approximately 60 and 80 kD. The differences in the apparent molecular weights of the degradation products at pH 4, 5.5 and 6 indicate different degradation processes at different pH values.

The formulations at pH 7.4 and 8 showed no further bands below the main band. In none of the formulations, an increase in the bands above the main band was observed, indicating that aggregation did not increase in the non-reduced SDS-PAGE during the 7-day storage at 40° C.

### b) Influence of pH and Additives

Various formulations were prepared and stored as described above under a). The following pH values and additives were used here:

| Formu- | | | | |
|---|---|---|---|---|
| lation | Buffer | Additive | | PBS |
| 6 | 35.7 mmol/l citrate pH 6 | 2.5% | mannitol | 2.73 mmol/l |
| 7 | 35.7 mmol/l citrate pH 6 | 43% | sucrose | 2.73 mmol/l |
| 8 | 35.7 mmol/l citrate pH 6 | 0.71% | glycine | 2.73 mmol/l |
| 9 | 35.7 mmol/l citrate pH 6 | 2.2% | L-arginine | 2.73 mmol/l |
| 10 | 3.2 mmol/l phosphate | 3.9% | mannitol | 2.73 mmol/l |
| | pH 7.4 | | | |
| 11 | 3.2 mmol/l phosphate | 7.1% | sucrose | 2.73 mmol/l |
| | pH 7.4 | | | |
| 12 | 3.2 mmol/l phosphate | 1.1% | glycine | 2.73 mmol/l |
| | pH 7.4 | | | |
| 13 | 3.2 mmol/l phosphate pH | 3.6% | L-arginine | 2.73 mmol/l |
| | 7.4 | | | |
| 14 | 97.3 mmol/l Tris pH 8 | 1.3% | mannitol | 2.73 mmol/l |
| 15 | 97.3 mmol/l Tris pH 8 | 2.1% | sucrose | 2.73 mmol/l |
| 16 | 97.3 mmol/l Tris pH 8 | 0.35% | glycine | 2.73 mmol/l |
| 17 | 97.3 mmol/l Tris pH 8 | 1.1% | L-arginine | 2.73 mmol/l |

For each of the formulations, osmolality was measured at time t=0 and the pH at 40° C. was measured at time t=4 weeks:

| | Osmolality | |
|---|---|---|
| Formulation | (Osmol/kg) | pH |
| 6 | 0.320 | 6.20 |
| 7 | 0.321 | 6.12 |
| 8 | 0.281 | 6.12 |
| 9 | 0.296 | 10.14* |
| 10 | 0.321 | 7.36 |
| 11 | 0.323 | 7.11 |
| 12 | 0.241 | 7.34 |
| 13 | 0.292 | 10.54* |
| 14 | 0.293 | 8.08 |
| 15 | 0.298 | 8.21 |
| 16 | 0.279 | 8.10 |
| 17 | 0.290 | 8.99 |

*pH adjusted prior to addition of arginine

In all formulations, the pH of the formulation corresponded to the one expected, except for the formulations containing L-arginine. The L-arginine formulations had a much higher pH in comparison with the expected pH, and this was caused by the action of the very basic amino acid L-arginine. The extremely high pH of the L-arginine formulations has to be taken into account when analyzing the stability results of the formulations.

All formulations, except for the formulations containing glycine at pH 7.4, were isotonic (=osmolality of between 0.270 and 0.330 osmol/kg). The glycine formulation at pH 7.4 was weakly hypotonic.

The various formulations were subjected to HP-SEC chromatography as described above. Some of the samples showed a change in peak shape (broadening or tailing of the peak). Although the shape of the peak was different, the entire peak area was interpreted as peak area of the dimer and used for determining the purity. Therefore, the purity of these peaks is probably an overestimate. The samples concerned were marked in the table with an asterisk (*).

The results show that none of the formulations in citrate buffer, pH 6, was stable at 40° C. At least a 20% loss of IL-15/Fc dimer was observed. None of the additives had a protective activity in the formulations with citrate buffer, pH 6. Likewise, none of the formulations containing L-arginine (high pH) was stable at 40° C. The formulations at pH 7.4 containing sucrose and glycine were stable at 40° C. for 3 weeks. The strong decrease in purity in the formulations with phosphate buffer at pH 7.4 and mannitol after 4 weeks is due to a microbial contamination. A slight but continuous decrease (from 85-70%) in purity was observed in the formulations with Tris buffer, pH 8. No difference between the excipients sucrose, mannitol or glycine was observed in the formulation with Tris buffer pH 8. The same was also observed for the formulations with citrate buffer at pH 7.4.

| | | t = 1 | t = 2 | t = 3 | t = 4 |
|---|---|---|---|---|---|
| Formulation | t = 0 | week | weeks | weeks | weeks |
| 6 | 85.97 | 79.48 | 72.24* | 58.65* | nt |
| 7 | 85.74 | 81.05 | 69.54* | 61.01* | nt |
| 8 | 85.75 | 77.86 | 69.74* | 57.21* | nt |
| 9 | 88.51 | 70.93 | 73.29* | 80.37* | nt |
| 10 | 85.57 | 85.37 | 86.88 | 88.71* | 67.05* |
| 11 | 85.58 | 84.98 | 84.74 | 85.82 | 81.64 |
| 12 | 85.68 | 85.25 | 85.17 | 86.41 | 82.50 |
| 13 | 90.77 | 86.95* | 90.03* | 75.01* | nt |
| 14 | 86.11 | 83.64 | 81.84 | 81.41 | 74.87 |
| 15 | 86.11 | 83.62 | 81.82 | 81.82 | 75.40 |
| 16 | 86.38 | 84.04 | 82.39 | 82.29 | 75.65 |
| 17 | 87.00 | 81.14 | 73.07 | 72.32* | nt |

*= Shape of the IL-15/Fc main peak had changed
nt = Not tested

The purity of the individual formulations was also checked by means of RP-HPLC. For this purpose, use was made of a Vydac-214TP54 column (250×4.6 mm, 5 μm) which was operated at 25° C. and a flow rate of 1 ml per minute. The eluent used was a linear gradient of 100% water containing 0.1% TFA to 100% acetonitrile containing 0.1% TFA. Detection was carried out at 214 nm. As in HP-SEC chromatography, the peak shape also changed. The samples affected are marked in the table again by an asterisk (*). In general, the RP-HPLC results indicate that the formulations at pH 6 and those containing L-arginine were not stable at 40° C. The formulations at pH 7.4 and 8 containing sucrose and glycine and at pH 8 containing mannitol are stable at 40° C. for at least 3 weeks. The strong decrease in purity of the formulation containing mannitol at pH 7.4 can be attributed to a microbial contamination.

| | | t = 1 | t = 2 | t = 3 | t = 4 |
|---|---|---|---|---|---|
| Formulation | t = 0 | week | weeks | weeks | weeks |
| 6 | 99.97 | 89.86* | 78.52* | nt | nt |
| 7 | 100 | 90.07* | 97.78* | nt | nt |
| 8 | nt | nt | nt | nt | nt |
| 9 | 99.26 | 99.94* | 97.97* | nt | nt |
| 10 | 99.53 | 99.22 | 100 | 94.23* | 62.03* |
| 11 | 99.31 | 99.48 | 100 | 99.47 | 96.85 |
| 12 | 99.97 | 100 | 99.95 | 99.94 | 96.52 |
| 13 | 99.84 | 91.69* | 73.37* | 88.74* | nt |
| 14 | 99.26 | 99.15 | 99.96 | 99.04 | 94.72 |
| 15 | 99.34 | 99.28 | 99.95 | 99.39 | 96.36 |
| 16 | nt | 100 | nt | 99.13 | 96.89 |
| 17 | nt | 78.22* | nt | 98.14* | nt |

*= Shape of the IL-15/Fc main peak had changed
nt = Not tested

The samples were furthermore studied by means of SDS-PAGE (reduced and non-reduced). Gels containing 3-8% Tris acetate were used for this purpose. Gel electrophoresis was carried out at 130 V, 110 mA, 23 W for 80 minutes, after in each case 0.5 μg of protein in 20 μl had been applied to the gel. The bands were visualized by means of silver staining. Bands above the IL-15/Fc main band indicated aggregation and those below the main band indicated degradation. In the samples stored at pH 6, degradation occurred after one week at 40° C., whereas at pH 8 increasing aggregation was observed after 2 weeks. The 2-week old formulations at pH 7.4 exhibited degradation and, after 3 weeks, both degradation and aggregation. The IL-15/Fc formulations at pH 7.4 or 8, containing sucrose or glycine, and at pH 8, containing mannitol, were stable at 40° C. for 2 weeks. Degradation in the formulation containing mannitol, pH 7.4, was again caused by a microbial contamination.

Six formulations (corresponding to the samples with pH 6, 7.4 and 8, containing in each case sucrose and glycine) were checked for their stability after 4 weeks and studied in a T-cell proliferation test in comparison with buffer without IL-15/Fc. To this end, biological activity of IL-15/Fc was checked by studying the proliferation-inhibiting action on IL-15-stimulated murine CTLL-2 cells as a function of the dose. For this purpose, the CTLL-2 cells were stimulated with a constant concentration of IL-15 to half maximum and incubated with increasing IL-15/Fc concentrations. T-Cell proliferation was detected by means of a calorimetric assay (XTT test from Roche). Half-maximum inhibition (ED₅₀) was calculated across the active dose range by means of linear progression. Each sample was measured in duplicate. The results were as follows:

| Formulation | ED₅₀ |
|---|---|
| IL-15/Fc in citrate buffer pH | 51 ± 13 ng/3 × 10⁴ seeded |
| 6 + sucrose | cells |
| IL-15/Fc in citrate buffer pH | 21.9 ± 10.2 ng/3 × 10⁴ seeded |
| 6 + cycline | cells |
| IL-15/Fc in phosphate buffer | 2.5 ± 1.7 ng/3 × 10⁴ seeded |
| pH 7.4 + sucrose | cells |
| IL-15/Fc in phosphate buffer | 4.2 ± 0.8 ng/3 × 10⁴ seeded |
| pH 7.4 + glycine | cells |
| IL-15/Fc in Tris buffer pH | 7.0 ± 0.5 ng/3 × 10⁴ seeded |
| 8 + sucrose | cells |
| IL-15/Fc in Tris buffer pH | 9.2 ± 3.8 ng/3 × 10⁴ seeded |
| 8 + glycine | cells |
| Phosphate buffer pH 7.4 + | No influence |
| sucrose | |
| Phosphate buffer pH 7.4 + | No influence |
| glycine | |
| Tris buffer pH 8 + sucrose | No influence |
| Tris buffer pH 8 + glycine | No influence |
| Citrate buffer pH 6 + sucrose | No influence |
| Citrate buffer pH 6 + glycine | No influence |
| Control: IL-15/Fc in PBS pH 7.4 | 12 ± 0.5 ng/3 × 10⁴ seeded |
| | cells |

These results show that IL-15/Fc was biologically active in all formulations, exhibiting the highest activity in phosphate buffer, pH 7.4, containing sucrose or glycine. A slightly lower activity which, however, was not significantly different, was observed at pH 8. In contrast to this, formulations stored at pH 6 resulted in a significantly lower IL-15/Fc bioactivity. The buffer formulation itself has no influence on the bioassay.

Some formulations were also tested for their stability by means of HPAEC-PAD glycosylation analysis. In this case, the proportion of disialo, trisialo and tetrasialo structures in the various formulations was studied after four-week storage at 40° C.:

| Formulation | Disialo (%) | Trisialo (%) | Tetrasialo (%) |
|---|---|---|---|
| 7 | 35.87 | 40.52 | 23.61 |
| 8 | 35.30 | 39.85 | 24.86 |
| 11 | 36.18 | 38.09 | 25.72 |
| 12 | 34.61 | 41.34 | 27.54 |
| 15 | 32.51 | 40.89 | 26.60 |
| 16 | 34.10 | 40.18 | 25.71 |
| Control | 33.93 | 41.12 | 27.59 |

As the table above shows, all formulations tested were comparable with respect to the relative proportions of di-, tri- and tetrasialo structures. The asialo and monosialo structures differed between the various formulations. Formulations with sucrose comprise a plurality of peaks at these specific retention times in comparison with formulations without sucrose and with the IL-15/Fc control. Glycine formulations and the IL-15/Fc control were comparable with respect to the relative amounts of sialo and monosialo structures. The difference between sucrose formulations and glycine formulations is probably caused by the presence of sucrose in the formulations, since the buffer of the control formulation (without IL-15/Fc), which contained sucrose, also comprised this extra peak at the specific retention times. It can generally be concluded that the IL-15/Fc formulation at pH 6, 7.4 or 8 in the presence of glycine or sucrose has no influence on the glycosylation properties of IL-15/Fc. Even after storage at 40° C. over 4 weeks, no change in the glycosylation pattern was observed.

### c) Conclusions

The stability studies with IL-15/Fc suggest that the stability of the formulation is highly pH-dependent in the pH 4-8 range. The stability decreases with lowering the pH within this range. Formulations at a pH around 10 are less stable than formulations at a pH around 8. The best stability was observed at pH 7.4 and pH 8. No specific differences in stability were found among IL-15/Fc formulations containing sucrose, mannitol or glycine as additive.

### Example 6

### Glycosylation Patterns of Various Clones

In a further experiment, the glycosylation patterns of IL-15/Fc fusion proteins having different levels of expression were studied. Clone K 146 was prepared using pcDNA3.1 with CMV promoter without intron, with all other clones being prepared using pMG10Ala7 with CMV promoter and intron A. The level of expression in clone K 146 was from approx. 3 to 5 pg of protein/(cell day), the level of expression in clones KN 10 and KN 13 was from approx 9 to 13 pg of protein/(cell*day), and finally, the level of expression in clones KN 110 and KN 120 was from approx. 30 to 50 pg of protein/(cell*day). The glycosylation data are summarized in the following table:

| | Proportion | K | KN | KN | KN | KN |
|---|---|---|---|---|---|---|
| Method | (%) | 146 | 10 | 13 | 110 | 120 |
| HPAEC-PAD; | NANA/Σ (NANA, | 53 | 57 | 50 | 55 | 50 |
| Asialo | NGNA, asialo N- | | | | | |
| | glycans | | | | | |
| | NANA/NGNA | 100:0 | 100:0 | 100:0 | 100:0 | 100:0 |
| HPAEC-PAD; | Asialo | 24 | 31 | 32 | 26 | 27 |
| native | Monosialo | 4 | 7 | 11 | 12 | 10 |
| | Disialo | 11 | 14 | 18 | 20 | 22 |
| | Trisialo | 27 | 22 | 21 | 25 | 22 |
| | Tetrasialo | 34 | 26 | 18 | 18 | 18 |
| Labelling of | Biantennary | 25-30 | | 25-30 | | 25-30 |
| biochip-bound | Tri/tetra- | 70-75 | | 70-75 | | 70-75 |
| glycoproteins | antennary | | | | | |
| with lectins | Sialylation/ | 70-80 | | 60 | | 60 |
| | antenna | | | | | |
| | Sialylation/ | >90 | | >90 | | >90 |
| | N-glycan | | | | | |
| | Gal α 1-3 Gal | 5-10 | | <5 | | 15-20 |

1. Process for purifying an IL-15/Fc fusion protein from a liquid composition, which process comprises:
a) applying the composition to an affinity chromatography column and eluting a first IL-15/Fc eluate from the column and
b) applying the eluate of step a) to an ion exchange chromatography column and eluting a second IL-15/Fc eluate from the column.

2. (canceled)

3. Process according to claim 1, comprising the additional step c):
c) applying the eluate of step b) to a gel filtration column or to a hydrophobic interaction chromatography column and eluting a third IL-15/Fc eluate from the column.

4. Process according to claim 1, in which the composition is filtered and/or concentrated prior to or after at least one of steps a) and b).

5. (canceled)

6. Process according to any of claim 1, in which the pH of the eluate of step a) is made acidic prior to step b).

7-9. (canceled)

10. Process according to claim 1, in which the IL-15/Fc fusion protein, after purification, has a purity of at least 90%.

11. Purified IL-15/Fc fusion protein, which has a purity of at least 90%.

12. Purified IL-15/Fc fusion protein according to claim 11, in which the average sialylation per N-glycan is at least 70%.

13. Purified IL-15/Fc fusion protein according to claim 11, in which the average sialylation per antenna is at least 50%.

14. Purified IL-15/Fc fusion protein according to claim 11, in which the N-glycolylneuraminic acid portion of the sum of N-acetylneuraminic acids, N-glycolylneuraminic acids and asialo-N-glycans is no more than 20%.

15. Composition comprising a purified IL-15/Fc fusion protein according to claim 11 and also excipients and additives.

16-17. (canceled)
